(19) United States
(12) Patent Application Publication
(10) Pub. No.: US 2021/0378786 A1
(43) Pub. Date: Dec. 9, 2021

(54) Dental implant instrument set and sinus curettes for vertically displacing sinus mucosa to form a space for bone augmentation

(71) Applicant: Hsiu-Kuo CHEN, New Taipei City (TW)

(72) Inventor: Hsiu-Kuo CHEN, New Taipei City (TW)

(21) Appl. No.: 16/894,928

(22) Filed: Jun. 8, 2020

(51) Int. Cl. A61C 1/08; A61C 1/12; A61C 8/00

(52) U.S. Cl. CPC A61C 1/084; A61C 8/0089; A61C 1/12

(57) ABSTRACT

A dental implant instrument set for vertically displacing sinus mucosa to form a space for bone augmentation comprises a bone drill and a sinus curette. The bone drill includes a drill bit for drilling upwardly a hole at a site of the alveolar bone where a tooth is lost, so as to expose sinus mucosa. The sinus curette includes a holding portion and a curette head portion. The curette head portion has one end provided with a turning part for separating from the alveolar bone and displacing the exposed sinus mucosa inward through the hole drilled by the drill bit, so as to form a space for bone augmentation above the site of the alveolar bone where the tooth is lost. The turning part is arranged to be at a first angle between 45 to 135 degrees with the curette head portion.

### BACKGROUND OF THE INVENTION

### Field of Invention

The present invention is related to a dental implant instrument set comprising a sinus curette, and more particularly, to a dental implant instrument set for vertically displacing sinus mucosa to form a space for bone augmentation, which comprises a bone drill used for drilling upwardly a hole at a site of the alveolar bone where a tooth is lost and an artificial dental root is to be implanted so as to expose sinus mucosa, and a sinus curette used for separating from the alveolar bone and displacing the exposed sinus mucosa inward through the drilled hole, so as to form a space for bone augmentation above the site of the alveolar bone where the tooth is lost.

### Prior Art

In recent years, with the rapid development of the dental implant technology, dental implant gradually replaces conventional dental treatments for tooth loss and becomes popular. The procedure of dental implant is to implant by means of surgery an artificial dental root into a site of the alveolar bone where a tooth is lost, and after the artificial dental root is fitted with the alveolar bone, a crown is capped on the artificial dental root. Since the artificial dental root is deeply implanted into the alveolar bone and fitted with the alveolar bone, its structural strength is sufficient for daily mastication. Therefore, the artificial tooth is comparable both in function and in appearance to a natural one.

As mentioned above, the artificial dental root is deeply implanted into the alveolar bone and fitted with the alveolar bone. If the alveolar bone is under a poor condition (such as alveolar atrophy), the implanted artificial dental root would be partly exposed, so that it would be unable to be sufficiently fitted with the alveolar bone, resulting in the failure of the dental implant surgery. Therefore, before the dental implant surgery is performed, it would be required to estimate the condition of the alveolar bone where a tooth is lost in advance, to determine whether the surgery for bone augmentation should be performed in advance.

Conventionally, bone augmentation is performed by a lateral window technique for sinus lift surgery. Please refer to FIG. 3 and FIGS. 5A and 5B. FIG. 3 is a cross-sectional schematic view showing the physiological anatomical structure of upper jaw and the relative positions of teeth A1, alveolar bone A2, gingiva A3, sinus cavity A4, and sinus mucosa A5. FIGS. 5A and 5B are schematic views showing the conventional lateral window technique for sinus lift surgery, which is performed on a site of the lateral gingiva where a tooth is lost. As shown in FIG. 5A, a gum flap A30 is formed by incising the lateral gingiva and is lifted to expose the alveolar bone A2 beneath the gum flap A30. And then, a bone drill is used to drill a hole A6 into the alveolar bone A2 to expose the sinus mucosa A5 beneath the alveolar bone A2. Finally, the exposed sinus mucosa is separated from the alveolar bone and is displaced inward to form a space to be filled with bone powder for bone augmentation.

### SUMMARY OF THE INVENTION

As for the problem that the alveolar bone is under poor condition, conventionally, the lateral window technique for sinus lift surgery is performed for bone augmentation. However, it is disadvantageous in that a relatively large-area wound or incision would be required for such a surgery that is performed on the site of the lateral gingiva beneath which a tooth is lost, and consequently significant discomfort would be caused for a patient, and the complexity of the surgery is also increased. Therefore, the present invention is aimed at how to decrease the wound area required for and simplify the complexity of the surgery for bone augmentation, and thus significantly reduce the discomfort for the patient.

An object of the present invention is to provide a dental implant instrument set for vertically displacing sinus mucosa to form a space for bone augmentation, where the bone augmentation is performed vertically and upward from a site of the alveolar bone where a tooth is lost and an artificial dental root is to be implanted, so that the surgery for bone augmentation and the surgery for dental implant are performed at the same site. In comparison with the conventional lateral window technique for sinus lift surgery, it is able to decrease the wound area required for the surgery, to reduce significantly the discomfort for a patient, and to simplify the complexity of the surgery.

In order to achieve the above-mentioned object, the present invention provides a dental implant instrument set for vertically displacing sinus mucosa to form a space for bone augmentation comprising a bone drill and a sinus curette. The bone drill includes a drill bit for drilling upwardly a hole at a site of alveolar bone where a tooth is lost and an artificial dental root is to be implanted, so as to expose sinus mucosa. The sinus curette includes a holding portion and a curette head portion. The curette head portion has one end provided with a turning part for separating from the alveolar bone and displacing the exposed sinus mucosa inward through the hole drilled by the drill bit, so as to form a space for bone augmentation above the site of the alveolar bone where the tooth is lost. The turning part is arranged to be at a first angle with the curette head portion, and the first angle is between 45 to 135 degrees.

In implementation, the first angle is 45, 90, or 135 degrees.

In implementation, the curette head portion is arranged to be at a second angle with the holding portion, and the second angle is between 120 to 170 degrees.

In implementation, the bone drill further includes a stop ring fitted over the drill bit; and the stop ring is detachably fitted over the drill bit for controlling the drill bit's penetration depth into the alveolar bone.

In implementation, the stop ring is further provided with a positioning piece for fixing the drill bit within the stop ring. In one embodiment, the positioning piece is provided at and passing through a wall of the stop ring and is movable along a direction perpendicular to the wall of the stop ring, so as to have the drill bit fixed within the stop ring or loosened therefrom.

In implementation, the dental implant instrument set for vertically displacing sinus mucosa to form a space for bone augmentation further comprises an auxiliary adjuster for adjusting the stop ring's position fitted over the drill bit of the bone drill. In one embodiment, the auxiliary adjuster includes: a plurality of channels of different lengths, where the plurality of channels is formed within the auxiliary adjuster, and each channel has an opening on the auxiliary adjuster's external surface; and at least one indicator, formed on the auxiliary adjuster's external surface for indicating at least one channel's length. Thereby, according to a desired penetration depth of the drill bit of the bone drill into the alveolar bone, the drill bit of the bone drill is placed into one channel with a length corresponding to the desired penetration depth, and the stop ring is fitted over the drill bit of the bone drill at a position adjacent to the opening of the channel, so as to control the drill bit's penetration depth into the alveolar bone.

The present invention also provides a sinus curette for vertically displacing sinus mucosa to form a space for bone augmentation, comprising a holding portion and a curette head portion having one end provided with a turning part, where the turning part is arranged to be at a first angle with the curette head portion, and the first angle is between 45 to 135 degrees, and the turning part is used for separating from the alveolar bone and displacing the exposed sinus mucosa inward through the hole drilled by the drill bit, so as to form a space for bone augmentation above the site of the alveolar bone where the tooth is lost.

In implementation, the curette head portion is arranged to be at a second angle with the holding portion, and the second angle is between 120 to 170 degrees.

The present invention will become more fully understood by reference to the following detailed description thereof when read in conjunction with the attached drawings.

### BRIEF DESCRIPTION OF THE DRAWINGS

FIGS. 1A to 1C are schematic views showing different examples of a sinus curette for vertically displacing sinus mucosa to form a space for bone augmentation according to the present invention.

FIG. 2 is a perspective view showing the use of a bone drill and an auxiliary adjuster of a dental implant instrument set for vertically displacing sinus mucosa to form a space for bone augmentation according to the present invention.

FIG. 3 is a cross-sectional schematic view showing the physiological anatomical structure of upper jaw.

FIGS. 4A and 4B are schematic views showing the use of the dental implant instrument set for vertically displacing sinus mucosa to form a space for bone augmentation according to the present invention.

FIGS. 5A and 5B are schematic views showing the conventional lateral window technique for sinus lift surgery.

### DETAILED DESCRIPTION OF THE INVENTION

The present invention provides a dental implant instrument set and a sinus curette for vertically displacing sinus mucosa to form a space for bone augmentation. The dental implant instrument comprises a bone drill and a sinus curette. The bone drill includes a drill bit for drilling upwardly a hole at a site of alveolar bone where a tooth is lost and an artificial dental root is to be implanted, so as to expose sinus mucosa. The sinus curette includes a holding portion and a curette head portion. The curette head portion has one end provided with a turning part for separating from the alveolar bone and displacing the exposed sinus mucosa inward through the hole drilled by the drill bit, so as to form a space for bone augmentation above the site of the alveolar bone where the tooth is lost. The turning part is arranged to be at a first angle with the curette head portion, and the first angle is between 45 to 135 degrees.

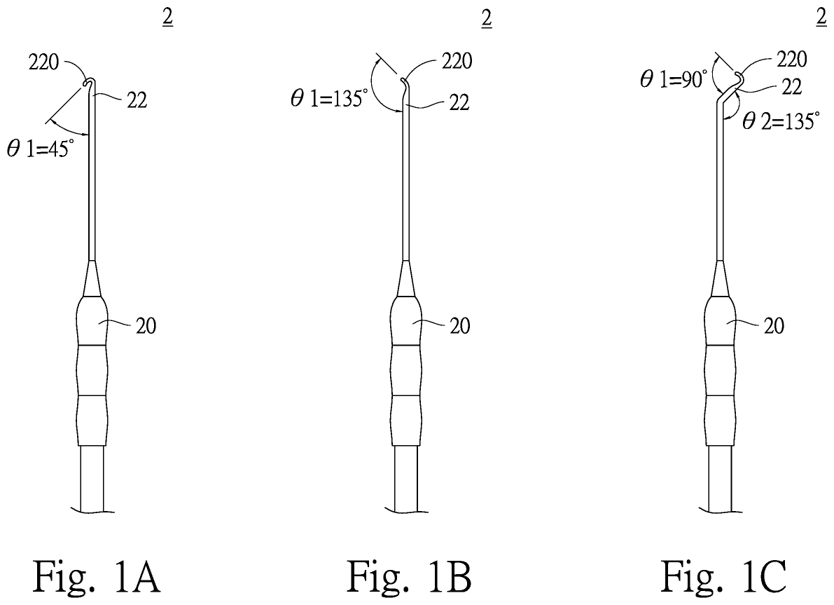

According to one embodiment of the present invention, the dental implant instrument set for vertically displacing sinus mucosa to form a space for bone augmentation comprises a bone drill 1, a sinus curette 2, and an auxiliary adjuster 3. Referring to FIGS. 1A to 1C, three different examples of the sinus curette are shown. As shown in FIG. 1A, the sinus curette 2 includes a holding portion 20 and a curette head portion 22. The curette head portion 22 has one end provided with a turning part 220. The turning part 220 is arranged to be at a first angle θ1 with the curette head portion 22, and the first angle θ1 is 45 degrees. As shown in FIG. 1B, the first angle θ1 between the turning part 220 and the curette head portion 22 is 135 degrees. As shown in FIG. 1C, the sinus curette 2 includes the holding portion 20, the curette head portion 22, and the turning part 220, where the first angle θ1 between the turning part 220 and the curette head portion 22 is 90 degrees, while the curette head portion is also arranged to be at a second angle θ2 with the holding portion, and the second angle θ2, which can be between 120 to 170 degrees, is 135 degrees in this embodiment.

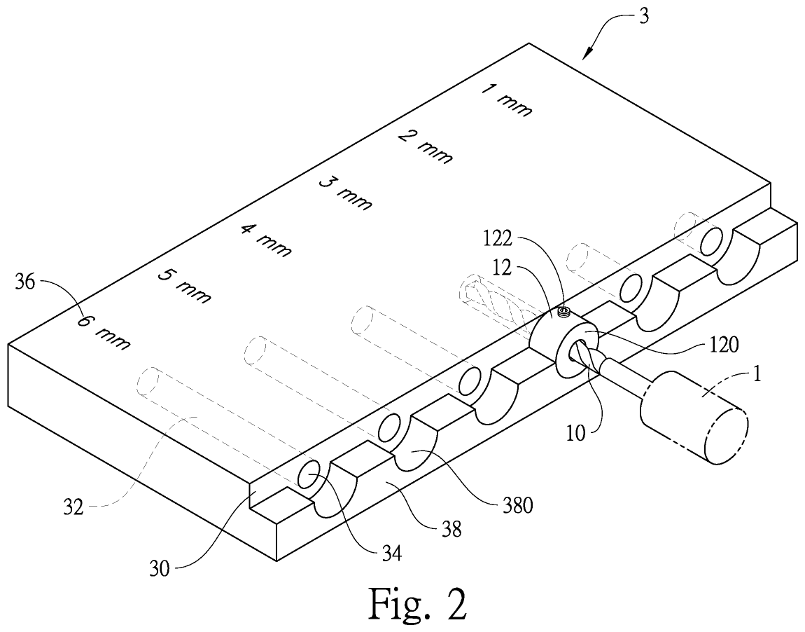

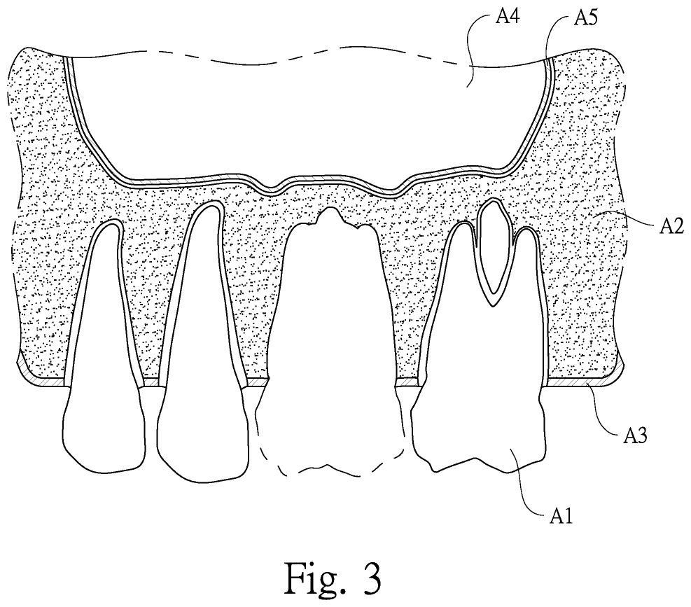

Referring to FIG. 2, it is a perspective view showing the use of the bone drill 1 and the auxiliary adjuster 3. The bone drill 1 includes a drill bit 10 and a stop ring 12 fitted over the drill bit 10. The stop ring 12 is detachably fitted over the drill bit 10 for controlling the penetration depth of the drill bit 1 of the bone drill 1 into the alveolar bone. Moreover, the stop ring 12 is further provided with a positioning piece 122 for fixing the drill bit 10 within the stop ring 12.

In this embodiment, the wall 120 of the stop ring 12 is provided with a thorough hole and the thorough hold has an internal surface provided with screw threads. The positioning piece is a screw capable of being engaged with the screw threads of the thorough hole. The screw is provided at and passing through the wall 120 of the stop ring 12 and is movable along a direction perpendicular to the wall 120 of the stop ring 12, so as to have the drill bit 10 fixed within the stop ring 12. In implementation, the positioning piece can be connected with the stop ring by way of clamping, elastic fastening, or magnetic fastening, in addition to screwing.

Moreover, as shown in FIG. 2, the auxiliary adjuster 3 is used for adjusting the position at which the stop ring 12 is fitted over the drill bit 10 of the bone drill 1. In this embodiment, the auxiliary adjuster 3 includes an external surface 30, a plurality of channels 32 of different lengths, openings 34 of these channels, indicators 36, and step portions 38. The plurality of channels 32 of different lengths is formed within the auxiliary adjuster 3. Each channel 32 has an opening 34 formed on the external surface 30 (lateral surface) of the auxiliary adjuster 3. The indicators 36 are arranged on the external surface 30 (top surface) of the auxiliary adjuster 3 for indicating the lengths of the channels. In this embodiment, the indicators and the openings of the channels are respectively provided on the top surface and the lateral surface. However, in implementation, they can be provided both on the lateral surface.

The step portions 38 of the auxiliary adjuster 3 are arranged to be adjacent to the openings 34 of the channels 32. Each step portion 38 is provided with a recessed groove 380 corresponding to the opening 34 of one channel 32 for receiving one stop ring 12 thereon. In implementation, it is first to choose out one channel 32 by an indicator that indicates the length corresponding to the desired penetration depth of the drill bit 10 of the bone drill 10 into alveolar bone. After the drill bit 10 is inserted into the channel 32, the stop ring 12 is fitted over the drill bit 10 at the position adjacent to the opening 34 of the channel 32 and is placed on the recessed groove 380 of the step portion 38. The positioning piece 122 is then used to fix the drill bit 10 within the stop ring 12. In this way, the stop ring can be fixed around the drill bit at the position in accordance with the desired penetration depth of the drill bit into alveolar bone.

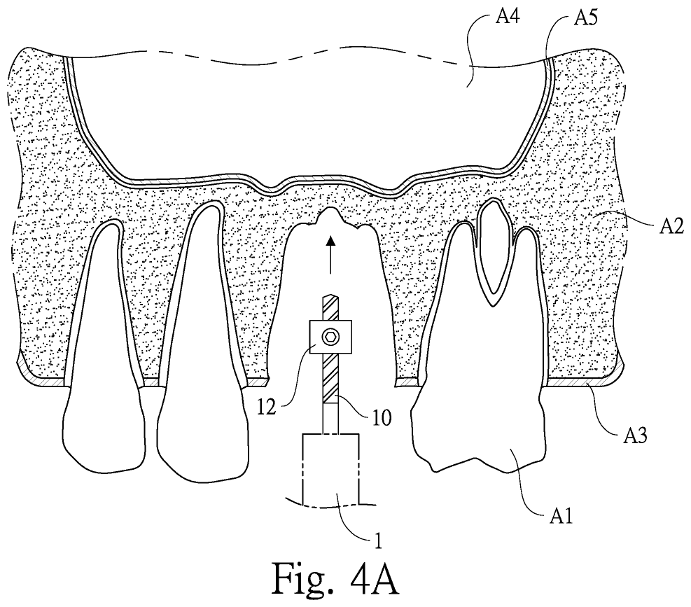

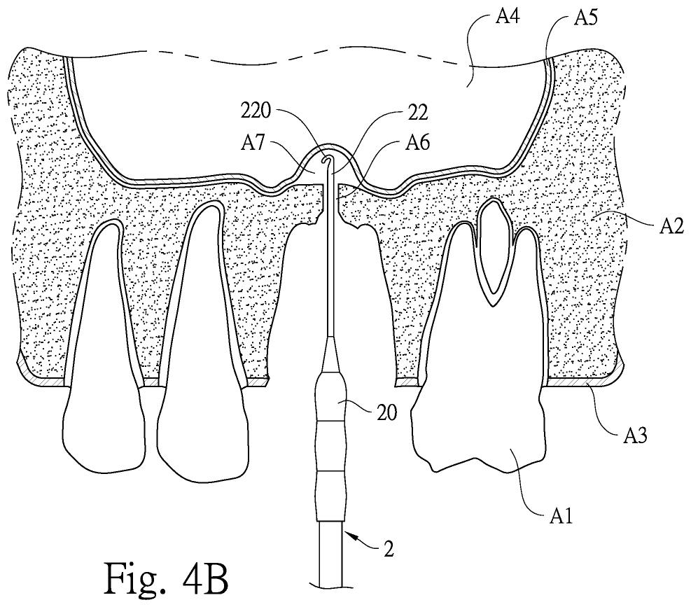

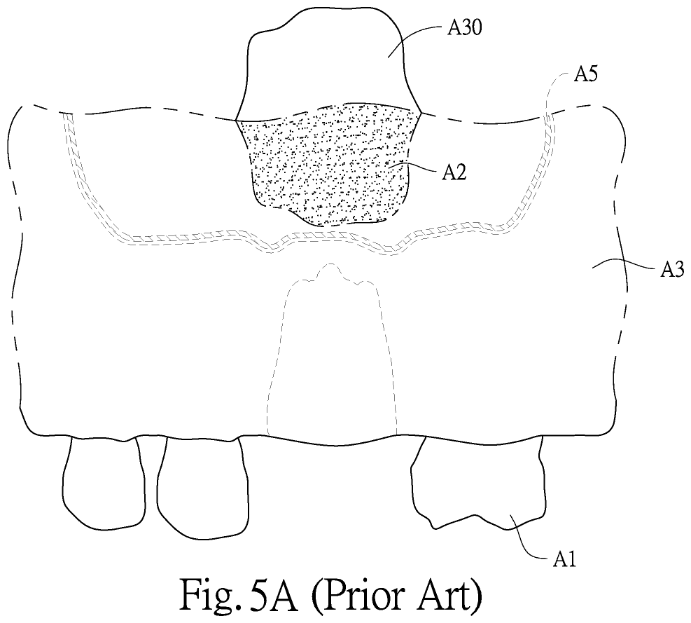

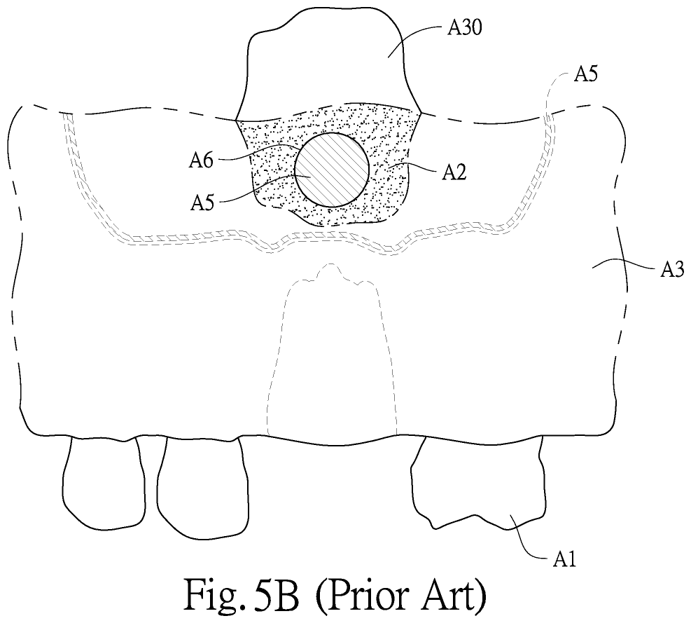

Refer to FIGS. 4A and 4B, which are schematic views showing the use of the dental implant instrument set for vertically displacing sinus mucosa to form a space for bone augmentation according to the present invention. Prior to the surgery, the desired penetration depth in an upward direction from the site where a tooth is lost and an artificial dental root is to be implanted (that is, the thickness of the alveolar bone beneath the sinus mucosa at this site) is determined by the results obtained via imaging techniques (such as the results obtained from X-ray examination). In accordance with this desired penetration depth, the stop ring 12 is fitted over the drill bit 10 of the bone drill 1 at a suitable position by using the auxiliary adjuster 3. During the surgery, as shown in FIG. 4A, the drill bit 10 of the bone drill 1 provided with the stop ring 12 is firstly used to drill upwardly a hole at a site of the alveolar bone A2 where a tooth is lost and an artificial dental root is to be implanted, so as to expose sinus mucosa A5. During the drilling process, a magnifier (not shown in the drawings) can be used to observe whether the sinus mucosa has been exposed.

Next, as shown in FIG. 4B, the turning part 220 of the sinus curette 2 is used to separate the sinus mucosa A5 from the alveolar bone A2 via the drilled hole A6, and then displace the sinus mucosa inward gradually to form a space for bone augmentation A7. In implementation, three different kinds of the sinus curettes, where the turning part is arranged to be at an angle of 45, 90, and 135 degrees with the curette head portion, can be used sequentially to separate from the alveolar bone and displace inward the exposed sinus mucosa. However, it is also feasible to use only one or two kinds of the sinus curettes according to the actual need.

Therefore, the present invention has following advantages:
1. According to the dental implant instrument set for vertically displacing sinus mucosa to form a space for bone augmentation of the present invention, a bone drill is used to drill upwardly a hole at a site of alveolar bone where a tooth is lost and an artificial dental root is to be implanted, so as to expose sinus mucosa. The surgery for bone augmentation performed upward from the site where the tooth is lost and the artificial dental root is to be implanted, is performed at the same site where the surgery for dental implant is performed. In comparison with the conventional lateral window technique for sinus lift surgery for bone augmentation, it is able to greatly decrease the wound area required for the surgery, to significantly reduce the discomfort for a patient, and to simplify the complexity of the surgery.
2. According to the dental implant instrument set for vertically displacing sinus mucosa to form a space for bone augmentation of the present invention, a sinus curette is used to separate from alveolar bone and displace inward the sinus mucosa. The turning part and the curette head portion of the sinus curette, or the curette head portion and the holding portion of the sinus curette, can be arranged to be at an angle with each other, so as to provide the sinus curette with much convenience or flexibility in use.
3. According to the dental implant instrument set for vertically displacing sinus mucosa to form a space for bone augmentation of the present invention, a stop ring is provided to be fitted over the drill bit for controlling the penetration depth of the drill bit into alveolar bone, while an auxiliary adjuster is provided for adjusting the position of the stop ring fitted on the drill bit of the bone drill, so as to enhance the safety.

In conclusion, according to above-disclosed descriptions, the present invention achieves the expected object to provide a dental implant instrument set and a sinus curette for vertically displacing sinus mucosa to form a space for bone augmentation. It is novel and has industrial use.

Although the embodiments of the present invention have been described in details, many modification and variations may be made by those skilled in the art from the teachings disclosed hereinabove. Therefore, it should be understood that any modification and variation equivalent to the spirit of the present invention be regarded to fall within the scope defined by the appended claims:

What is claimed is:

1. A dental implant instrument set for vertically displacing sinus mucosa to form a space for bone augmentation, comprising:
a bone drill, including a drill bit for drilling upwardly a hole at a site of alveolar bone where a tooth is lost and an artificial dental root is to be implanted, so as to expose sinus mucosa; and
a sinus curette, including a holding portion and a curette head portion, where the curette head portion has one end provided with a turning part for separating from the alveolar bone and displacing the exposed sinus mucosa inward through the hole drilled by the drill bit, so as to form a space for bone augmentation above the site of the alveolar bone where the tooth is lost; and the turning part is arranged to be at a first angle with the curette head portion, and the first angle is between 45 to 135 degrees.

2. The dental implant instrument set for vertically displacing sinus mucosa to form a space for bone augmentation according to claim 1, wherein the first angle is 45, 90, or 135 degrees.

3. The dental implant instrument set for vertically displacing sinus mucosa to form a space for bone augmentation according to claim 1, wherein the curette head portion is arranged to be at a second angle with the holding portion, and the second angle is between 120 to 170 degrees.

4. The dental implant instrument set for vertically displacing sinus mucosa to form a space to be filled with bone regeneration material according to claim 1, wherein the bone drill further includes a stop ring fitted over the drill bit; and the stop ring is detachably fitted over the drill bit for controlling the drill bit's penetration depth into the alveolar bone.

5. The dental implant instrument set for vertically displacing sinus mucosa to form a space for bone augmentation according to claim 4, wherein the stop ring is further provided with a positioning piece for fixing the drill bit within the stop ring.

6. The dental implant instrument set for vertically displacing sinus mucosa to form a space for bone augmentation according to claim 5, wherein the positioning piece is provided at and passing through a wall of the stop ring and is movable along a direction perpendicular to the wall of the stop ring, so as to have the drill bit fixed within the stop ring or loosened therefrom.

7. The dental implant instrument set for vertically displacing sinus mucosa to form a space for bone augmentation according to claim 4, further comprising an auxiliary adjuster for adjusting the stop ring's position fitted over the drill bit of the bone drill.

8. The dental implant instrument set for vertically displacing sinus mucosa to form a space for bone augmentation according to claim 7, wherein the auxiliary adjuster includes:
a plurality of channels of different lengths, where the plurality of channels is formed within the auxiliary adjuster, and each channel has an opening on the auxiliary adjuster's external surface; and
at least one indicator, formed on the auxiliary adjuster's external surface for indicating at least one channel's length;
thereby, according to a desired penetration depth of the drill bit of the bone drill into the alveolar bone, the drill bit of the bone drill is placed into one channel with a length corresponding to the desired penetration depth, and the stop ring is fitted over the drill bit of the bone drill at a position adjacent to the opening of the channel, so as to control the drill bit's penetration depth into the alveolar bone.

9. A sinus curette for vertically displacing sinus mucosa to form a space for bone augmentation, comprising a holding portion and a curette head portion having one end provided with a turning part, where the turning part is arranged to be at a first angle with the curette head portion, and the first angle is between 45 to 135 degrees, and the turning part is used for separating from the alveolar bone and displacing exposed sinus mucosa inward through the hole drilled by a drill bit, so as to form a space for bone augmentation above a site of alveolar bone where a tooth is lost.

10. The sinus curette for vertically displacing sinus mucosa to form a space for bone augmentation according to claim 9, wherein the curette head portion is arranged to be at a second angle with the holding portion, and the second angle is between 120 to 170 degrees.
